(19) United States
(12) Patent Application Publication
(10) Pub. No.: US 2014/0128336 A1
(43) Pub. Date: May 8, 2014

(54) MEDICAL PRODUCTS FOR USE IN CONDITIONS RELATED TO MICROBIAL INFECTIONS IN THE UPPER AERODIGESTIVE TRACT

(71) Applicants: Matti Härkönen, Espoo (FI); Tuuli Marvola, Helsinki (FI); Mikko Salaspuro, Helsinki (FI); Osmo Suovaniemi, Helsinki (FI)

(72) Inventors: Matti Härkönen, Espoo (FI); Tuuli Marvola, Helsinki (FI); Mikko Salaspuro, Helsinki (FI); Osmo Suovaniemi, Helsinki (FI)

(73) Assignee: BIOHIT OYJ, Helsinki (FI)

(21) Appl. No.: 14/111,913

(22) PCT Filed: Apr. 18, 2012
(86) PCT No.: PCT/FI2012/050377

§ 371 Date: Jan. 3, 2014

(30) Foreign Application Priority Data
Apr. 18, 2011 (FI) 20115377

(51) Int. Cl. A61K 31/198; A61K 31/5383; A61K 31/4164; A61K 31/7048; A61K 31/43; A61K 31/65

(52) U.S. Cl. CPC A61K 31/198; A61K 31/43; A61K 31/65; A61K 31/4164; A61K 31/7048; A61K 31/5383
USPC 514/29; 514/562; 514/197; 514/154; 514/398; 514/230.2

(57) ABSTRACT

The present invention concerns products, such as compositions or medical devices, comprising one or more compounds of the Formula I, containing one or more free sulphhydryl groups,

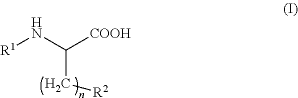

where
R¹ is hydrogen or an acetyl group, and
R² is a sulphhydryl group or a C1-C5 straight chained or branched hydrocarbon chain, optionally containing one or more heteroatoms selected from O, N and S, further containing one or more free sulphhydryl groups,
for use in connection with Helicobacter pylori infections or other related infections, either as such or as combinations with antibiotics, whereby the compound(s) and optionally, when present, the antibiotic(s), are mixed with one or more pharmaceutically acceptable carriers providing sustained local release in the stomach or elsewhere in the upper aerodigestive tract.

### FIELD OF THE INVENTION

The present invention concerns pharmaceutically acceptable compositions, medical devices and combinations for partly or completely destroying biofilms formed by Helicobacter pylori or other microbes that are able to survive in the upper aerodigestive tract, especially in the stomach, or several such microbes, or for preventing such biofilm formation, or for eradicating the microbe(s).

### DESCRIPTION OF RELATED ART

Helicobacter pylori (H. pylori) infection is a cause of chronic active gastritis, which significantly enhances the risk for intestinal metaplasia in the stomach, and is undoubtedly involved in gastric carcinogenesis. H. pylori infection is as well the main pathogenetic factor leading to atrophic gastritis that is the main risk factor for stomach cancer. H. pylori play a crucial role in the pathogenesis of peptic ulcer and its complications, such as bleeding and stenosis. Furthermore, H. pylori are the main pathogenetic factor behind mucosa-associated lymphoid tissue lymphoma. In addition to H. pylori, many other microbes may also cause prolonged and sometimes severe infections in the upper aerodigestive tract.

According to the Maastricht 2 guidelines the first-line treatment for H. pylori eradication is the triple therapy using a proton-pump inhibitor (PPI), amoxicillin, and clarithromycin (1). Caused by the wide spread use of antibiotics in general practice, the resistance to antibiotics, such as clarithromycin has increased world-wide. This is associated with a corresponding decrease in the eradication rate for H. pylori infection. Furthermore, cross-resistance between antibiotics appears. Increasing incidence of antibiotic resistance is the main and increasing cause of failure in H. pylori eradication (2). This results in multiple treatments leading to increasing number of side effects and increasing cost.

It is well known that Helicobacter pylori are able to survive and multiply in the gastric mucosa of the acidic stomach due to their urease activity and ability to secrete ammonium bicarbonate and thus strengthen the mucus-bicarbonate barrier that protects both Helicobacter pylori and gastric mucosa from acid and pepsin.

Several studies have shown the ability of Helicobacter pylori to form biofilm (3, 4). The possible survival of biofilm outside the gastric niche has been suggested as a probable transmission mode for this organism (5). Two distinct studies have shown the presence of biofilm on the gastric mucosa of H. pylori-positive subjects, as well as the absence of biofilm in H. pylori-negative control subjects (6, 7). Biofilm demolition has also been attempted (8), but only using immediate-release formulations that have a short term effect in the stomach, since they are rapidly transported further to the small intestine after administration, whereby they will not have a long period of action in the upper gastrointestinal tract and will, therefore, not provide the desired efficiency in problematic cases, which demand an ideal efficiency in biofilm demolition and ideal conditions for the eradication of H. pylori and other biofilm forming microbes.

In a study it has been found that the growth of Cladosporium cladosporioides was completely and statically inhibited when it was cultured with cysteine (9). Also the production of ATP in C. cladosporioides was inhibited by cysteine. When a silicone block was incubated with C. cladosporioides, the surface of the block was coated with the biofilm of C. cladosporioides. However, the block containing cysteine was not covered with biofilm. The same has not been previously demonstrated with H. pylori or other microbes of the upper aerodigestive tract, nor with other active compounds than cysteine. Further, this study shows no relation to the demolition of biofilm.

Atrophic gastritis is a well-known risk factor for gastric cancer. One factor contributing to this risk is the condition achlorhydria, which leads to microbial colonization of the stomach. Several of these formed microbes are able to produce significant amounts of acetaldehyde by oxidation from ingested alcohol (10). Under anaerobic conditions these microbes are able to produce acetaldehyde also from glucose. Acetaldehyde present in alcoholic beverages and formed from ethanol endogenously has recently been classified as a group one carcinogen in humans. Acetaldehyde can be eliminated from saliva after alcohol intake and during smoking with a semi-essential amino acid, cysteine, particularly in the form of L-cysteine (11, 12). More specifically, it has been shown that a formulation releasing for example L-cysteine in a controlled manner (e.g. the formulation known as Acetium®) can be used to decrease the acetaldehyde concentration during alcohol exposure in order to minimize the exposure to carcinogenic acetaldehyde (ref. 13, and EP 01980581). However, no link has been shown between the use of cysteine and the formation of biofilm.

Thus, there is a need for novel therapeutic treatments that would be capable of demolishing the biofilm formed by Helicobacter pylori (H. pylori) or other microbes in the gastric mucosa of the stomach, and potentially even eradicate these microbes, while making them susceptible to gastric acid or reducing their resistance to various antibiotics.

### SUMMARY OF THE INVENTION

It is an aim of the present invention to provide pharmaceutically acceptable products suitable for use in improved treatments of conditions related to Helicobacter pylori infections.

Particularly, it is an aim of the present invention to provide compounds used as the only active agents in compositions or devices, or in products intended for combination treatments to give controlled-release formulations suitable for use in improved treatments of Helicobacter pylori and other infections, and conditions caused by these infections.

These and other objects, together with the advantages thereof over known products and methods, are achieved by the present invention, as hereinafter described and claimed.

The invention is based on the finding that mucolytic pretreatment with cysteine or cysteine derivative is able to demolish the biofilm architecture, rendering H. pylori strains susceptible to acid and pepsin attack, as well as more vulnerable to antibiotics.

These agents are able to act as biofilm dissolving agents or as agents preventing its formation, thus sensitizing H. pylori to acid, pepsin and antibiotic action, possibly by demolishing not only the biofilm formed by Helicobacter pylori but also in part the mucus layer that protects Helicobacter pylori from the acid and pepsin in the stomach. In fact, in optimum conditions, the Helicobacter pylori can become eradicated even without the optional antibiotic regimen aimed for H. pylori eradication.

Thus, formulations releasing cysteine, N-acetylcysteine or another biodegradable, non-toxic agent containing free sulphhydryl groups, preferably selected from sulphhydryl group—containing amino acids, more preferably from cysteine, N-acetylcysteine or another derivative of cysteine, most suitably from N-acetylcysteine and L-cysteine, in a controlled, preferably slow, manner and locally in the stomach can be used as a first line treatment form for Helicobacter pylori infection. These formulations may also contain and release other agents, particularly ones suggested to be able to prevent microbial biofilm formation.

According to one hypothesis, the mechanism by which cysteine, N-acetylcysteine or another derivative of cysteine prevents the biofilm formation of Helicobacter pylori or demolishes them is by destroying the sulfur double bonds of the thiol (SH) group of the film-forming agents and thus acting as a biofilm dissolving agent. This mucolytic property of the agent sensitizes H. pylori to acid and pepsin attacks and as well as to antibiotic action.

Formulations releasing, for example, cysteine or N-acetylcysteine in a controlled manner are able to eradicate Helicobacter pylori even as a mono therapy without any antibiotics and the associated risk of developing resistant bacteria.

The “products” of the present invention are meant to include compositions containing one or more compounds of the Formula I, medical devices capable of carrying one or more of these compounds of Formula I to the desired site of action, as well as combination products containing one or more of the compounds of Formula I together with one or more antibiotics, either in the same formulation or administered separately. Each of these products can be used together with, or can include, one or more further active agents.

Thus, the present invention concerns compositions comprising one or more compounds of the Formula I, containing one or more free sulphhydryl groups,

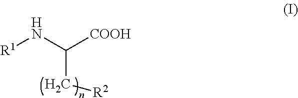

where
R¹ is hydrogen or an acetyl group, and
R² is a sulphhydryl group or a C1-C5 straight chained or branched hydrocarbon chain, optionally containing one or more heteroatoms selected from O, N and S, further containing one or more free sulphhydryl groups,
for use in connection with Helicobacter pylori infections or other related infections, either as such or as combinations with antibiotics.

Further, the present invention concerns medical devices in the form of monolithic or multiparticular tablets or capsules, or granules as such, or having the physical structure of a gel.

More specifically, the composition of the present invention is characterized by what is stated in Claim 1.

Further, the medical device of the present invention is characterized by what is stated in Claim 15 and the combinations of the present invention are characterized by what is stated in Claim 18.

Considerable advantages are obtained by means of the invention. Thus, the present invention provides controlled release of the compounds of Formula I, resulting in a local effect of this active ingredient in the stomach. This, in turn provides new effective treatment(s) for conditions relating to Helicobacter pylori infections or other related infections. These new and effective treatments do not necessarily include any use of antibiotics, as the Helicobacter pylori or other bacteria can become eradicated even without an antibiotic regimen, or alternatively a less violent antibiotic regimen can be used. Thus, the present invention will eventually result in a reduction of antibiotic resistance. Further, the invention reduces the need for multiple treatments in these conditions relating to bacterial infections in the upper aerodigestive tract.

Next, the invention will be described more closely with reference to the following drawings, a detailed description and examples.

### DESCRIPTION OF THE DRAWINGS

FIG. 1 is a series of pictures illustrating the formation of a biofilm, in vitro, using H. pylori.

FIG. 2 is a series of pictures illustrating the formation of biofilm, in vitro, using H. pylori, with the bottom pictures illustrating the inhibition of biofilm using 10 mg/ml and 20 mg/ml of N-acetylcysteine, as compared to 2 mg/ml of N-acetylcysteine.

FIG. 3 is a series of pictures illustrating the formation of biofilm, in vitro, using H. pylori, with the bottom pictures illustrating the inhibition of biofilm using 10 mg/ml and 20 mg/ml of L-cysteine, as compared to 2 mg/ml of L-cysteine.

### DETAILED DESCRIPTION OF THE PREFERRED EMBODIMENTS OF THE INVENTION

The present invention concerns compositions comprising one or more compounds of the Formula I, containing one or more free sulphhydryl groups,

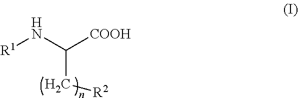

where
R¹ is hydrogen or an acetyl group,
R² is a sulphhydryl group or a C1-C5 straight chained or branched hydrocarbon chain, optionally containing one or more heteroatoms selected from O, N and S, further containing one or more free sulphhydryl groups, R² preferably being a sulphhydryl group, and
n is an integer of 1 to 3, preferably being 1,
for use in connection with Helicobacter pylori or other related infections, either as such or as combinations with antibiotics, whereby the compound(s) and optionally, when present, the antibiotic(s), are mixed with one or more pharmaceutically acceptable carriers providing sustained local release in the stomach.

The term “use in connection with Helicobacter pylori infections or other related infections” is intended to encompass use in partly or completely destroying biofilms formed by Helicobacter pylori or other microbes that are able to survive in the upper aerodigestive tract, or several such microbes, or for preventing such biofilm formation, or for eradicating the microbe(s).

The term “upper aerodigestive tract” is intended to encompass the mouth, the pharynx, the esophagus and the stomach, particularly the stomach.

Particularly, the compound(s) or combination(s) are for use in partly or completely destroying biofilms formed by Helicobacter pylori or other microbes that are able to survive in the stomach and upper aerodigestive tract, or several such microbes, or for preventing such biofilm formation, or for eradicating the biofilm-forming microbe(s).

Said other microbes that are able to survive in the stomach and upper aerodigestive tract include microbes commonly present in the esophagus and in the mouth, including some species of the genus Candida as well as dental bacteria.

The compounds of Formula I are preferably selected from cysteine and its derivatives and mixtures thereof, more preferably from cysteine or N-acetylcysteine, most suitably from L-cysteine.

The composition may contain also other active agents, e.g. lipolytic antibiotics, chelating agents and alpha-hydroxy acids (HICAs) suggested to be able to hamper/demolish microbial biofilms, or acid, such as betaine hydrochloride or glutamic acid, or pepsin or both, to relieve any conditions related to an achlorhydric stomach, or to further prevent a potential conversion of cysteine to the less reactive cystine.

However, according to a preferred embodiment of the invention, the active agent(s) of the present composition are limited to the compounds of Formula I, optionally in combination with one or more antibiotics.

According to a particular aspect of the invention, only natural compounds are used as the active agent(s).

According to another particular aspect, the use of metallic compounds (containing metal ions) is avoided due to their reactivity and the inclination of some of these to cause harm, such as dyspeptic problems.

Further, the present invention concerns a medical device in the form of monolithic or multiparticular tablets or capsules, or granules as such, or having the physical structure of a gel. This medical device functions by delivering the active agent(s) to its(their) desired site of action.

Said device contains one or more pharmaceutically acceptable carriers providing sustained local release in the stomach, the device being filled or mixed with a composition comprising one or more compounds of the Formula I, containing one or more free sulphhydryl groups,

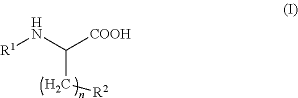

where
R¹ is hydrogen or an acetyl group,
R² is a sulphhydryl group or a C1-C5 straight chained or branched hydrocarbon chain, and
n is an integer of 1 to 3, preferably being 1,
optionally containing one or more heteroatoms selected from O, N and S, further containing one or more free sulphhydryl groups, R² preferably being a sulphhydryl group, optionally mixed with one or more further pharmaceutically acceptable carriers.

The composition, combination or device preferably contains one or more compounds of Formula I in an amount of 50-500 mg, more preferably 50-300 mg, and most suitably 100-200 mg. This amount is suitable for partly or even completely destroying said biofilm. Preferably, a sufficient amount of the compound(s) of Formula I is administered to the subject to completely destroy said biofilm.

A single dose (unit dose) of the composition may comprise 50-500 mg, preferably 50-300 mg, and most suitably 100-200 mg of the active compound(s) of Formula I, which dose can be provided using one or more formulations. In this context, the “formulation” is intended to include one tablet, capsule, lozenge or chewing gum, or one unit of a gel or a gel-forming liquid.

The optional further active agent(s) (such as HICAs, acid or pepsin) may be added to the formulation in amounts of 1 to 50 weight-% of the total amount of active agent(s), preferably 10 to 40 weight-%, most suitably 20 to 30 weight-%.

Most suitably, the compounds, compositions or combinations are formulated using suitable carriers into controlled-release formulations. The term “carrier” is intended to include also fillers and binders.

According to one preferred alternative, the used carriers are selected from the carriers that are capable of maintaining an effective concentration of the pharmaceutically active compound in the stomach for preventing the biofilm formation capacity of Helicobacter pylori during a period of 30 minutes.

According to another preferred alternative, the carrier(s) are selected from the carriers that are capable of providing an effective concentration of the pharmaceutically active compound in the stomach for at least partly destroying the biofilm formed on the surface of gastric mucosa by Helicobacter pylori or any other microbes that are able to survive in the stomach or upper aerodigestive tract.

According to a third preferred alternative, the carrier(s) are selected from the carriers that are capable of providing an effective concentration of the pharmaceutically active compound in the stomach for eradicating Helicobacter pylori, or any other microbes that without treatment are able to survive in the stomach or upper aerodigestive tract, when used as a mono therapy in subjects infected with such microbes, or that are capable of causing an enhanced eradication rate of such microbes.

Said subjects may be either human or animal patients.

According to a particularly preferred embodiment, the used carriers are selected from those that are capable of controlling the releasing speed of the compounds of Formula I so that these compounds are released, locally, in the stomach during a period of 60-120 minutes.

Examples of such carriers are substances, which are selected from the group of various chitosans, alginates, such as sodium alginate, aluminium hydroxide, sodium carboxymethyl cellulose, sodium hydrogen carbonate, and enteric polymers, preferably from enteric polymers. Such carriers may be used either alone or as combinations of two or more substances.

Thus, the composition according to the invention, which releases its contents in the stomach, contains at least one—preferably two—polymers, in the form of additives, such as carriers, fillers or binders, which have the task of keeping the drug as long as possible, for two hours minimum, in the stomach so that it forms a gel that floats in the contents of the stomach. Another task of the polymers is to prolong the release of the effective substance(s).

The composition is preferably in the form of an encapsulated composition comprising a mixture of powder or granules that form a gel in the stomach. In addition to the cysteine, the composition comprises said polymers and optional further additives. This encapsulated composition is most suitably formulated to be swallowed by the subject.

The amount of polymers in this composition is 10-50%, preferably 15-40%, and most preferably 20-30%.

According to an alternative, the cysteine in the composition is mixed with the fillers needed and, after that, granulated by using enteric polymers as binders.

Thus, according to this alternative, the formulation may include, for example granules contained in an HPMC capsule, the granules containing a suitable filler and a suitable binder, as well as, optionally, further conventional pharmaceutical additives. An exemplary granule composition may contain:

| | |
|---|---|
| L-Cysteine or N-acetylcysteine | 100 mg |
| Calcium hydrogen phosphate | 30-50 mg |
| Eudragit RS-PO | 40-60 mg |
| titanium dioxide | 5-10 mg |

According to another particularly preferred embodiment, the used carriers are selected from those that are capable of controlling the releasing speed of the compounds of Formula I so that these compounds are released, locally, in the mouth during a period of 60-120 minutes. Thus, the released contents will have an effect not only in the mouth, but also in the areas of the aerodigestive tract to where they are carried, e.g. with the saliva, these areas including the pharynx, the esophagus and the stomach.

Examples of such carriers are substances, which are selected from the group of various chitosans, alginates, such as sodium alginate, aluminium hydroxide, sodium carboxymethyl cellulose, and sodium hydrogen carbonate. Such carriers may be used either alone or as combinations of two or more substances.

Thus, the composition according to the invention, which releases its contents in the mouth, contains at least one—preferably two—polymers, in the form of additives, such as carriers, fillers or binders, which have the task of keeping the drug as long as possible, for two hours minimum, in the mouth so that it forms a gel that adheres to the mucous membranes. Another task of the polymers is to prolong the release of the effective substance(s).

The composition is preferably in the form of a tablet, capsule, lozenge or chewing gum, forms a gel when placed in contact with the saliva. In addition to the cysteine, the composition comprises said polymers and optional further additives. This composition is most suitably formulated to be kept in the mouth, by the subject placing it under the tongue, sucking on it or chewing on it.

The amount of polymers in this composition is 10-50%, preferably 15-40%, and most preferably 20-30%.

In general, the formulation according to the invention preferably contains filler in the form of calcium hydrogen phosphate (CaHPO₄), which has the advantages of not swelling in the stomach content and of being suitable for direct compression. The amount of filler in the formulation is preferably 30-70%, most suitably 40-60%.

Further, the formulation preferably contains binder selected from any known enteric polymer, preferably a methacrylate derivative, more preferably a derivative known by the trade names Eudragit, and most suitably Eudragit RS-PO. The amount of enteric polymer in the formulation is preferably 2-5%, most preferably 3-4% of the entire formulation.

The formulation can also contain other conventional additives, such as titanium dioxide, preferably in minor amounts of 2-5% of the entire formulation.

In case of using polymers as carriers, it is particularly preferred to mix the polymer(s) or the compound(s) of Formula I or the antibiotic(s) of a combination with 10-30%, preferably 20% sodium hydrogen carbonate of the weight of the polymer(s).

The total amount of polymers in the composition or the combination or the device is then, generally, 10-50%, preferably 15-40%, and most preferably 20-30% of the weight of the composition or the combination or the device.

The product of the invention (i.e. the composition or combination or device) is for use in partly or completely destroying biofilms formed by Helicobacter pylori or other microbes that are able to survive in the stomach or elsewhere in the upper aerodigestive tract, or several such microbes, or for preventing such biofilm formation, or for eradicating the microbe(s), particularly in patients carrying said microbe(s) that are sensitive to acid or resistant to one or more antibiotics that are used in the conventional treatment of an infection by said microbe(s).

Said product is preferably in the form of monolithic or multiparticular tablets or capsules, or granules as such, or it has the physical structure of a gel. This product is intended to be administered to Helicobacter pylori infected patients daily for a period of from 3 days to 4 weeks. Using the products of the present invention, a long-lasting treatment can be used, optionally with the mere intention to ensure a complete demolition of biofilm or eradication of harmful bacteria, since the product of the invention contains no substances that in the used concentrations and amounts would be harmful or toxic.

However, an effective treatment or prevention of conditions related to H. pylori infections or other microbial infections can be obtained using a period of daily administration of considerably less than 4 weeks, such as from 3 days to 3 weeks, or 3 to 14 days, or even 3 to 7 days.

A product intended to be swallowed by the subject, to be carried to the stomach, is suitably in the form of a tablet or a capsule, particularly comprising a mixture of powder or granules. Most preferably, it is in the form of capsules comprising granules.

In case of the product being in the form of granules (optionally contained in capsules), these granules preferably contain, as binders, enteric polymers, preferably methacrylate derivatives, the solution pH of which is 6-7 and the total amount of the weight of the preparation is 2-5%, preferably 3-4%.

According to a preferred embodiment of the present invention, the product is administered to a patient using a dose of 50-300 mg, preferably 100-200 mg, between meals, such as at 3- to 6-hour intervals, preferably at around 4-hour intervals, 2-6 times a day, preferably 4-6 times a day, most suitably to an empty stomach, for example at least 1 hour, preferably at least 2 hours, or within 2-5 hours, after the previous meal.

For eradication of the harmful microbes of the upper aerodigestive tract, an effective alternative is to administer a single dose, or possibly even a 2-4 times larger dose than the above mentioned one, just prior to bedtime, to allow the active agent to react while the subject is asleep.

In case of a combination product, i.e. a formulation intended for combination therapy, the product (i.e. the formulation) comprises one or more antibiotics, either mixed with the compound(s) of Formula I, or administered separately in a manner providing at least partial overlap of the period of action of the compound(s) of Formula I and the antibiotic(s). This administration provides the desired synergy.

The antibiotics are preferably selected from the group of amoxicillin, ampicillin, clarithromycin, metronidazole, tetracyclin and levofloxacin.

More preferably, the antibiotic regimen is selected from the commonly used alternatives, such as one of the following:

1) L-cysteine/N-acetylcysteine as a monotherapy for 1-4 weeks
2) L-cysteine/N-acetylcysteine+amoxicillin 1000 mg×2 or tetracycline 500 mg×4 for 1-2 weeks
3) L-cysteine/N-acetylcysteine+amoxicillin 1000 mg×2+metronidazole or tinidazole 500 mg×2+Claritromycin 500 mg×2
4) L-cysteine/N-acetylcysteine+amoxicillin 1000 mg×2+metronidazole or tinidazole 500 mg×2+Claritromycin 500 mg×2+PPI (protein pump inhibitor) standard dose×2
5) L-cysteine/N-acetylcysteine+amoxicillin 1000 mg×2 days 1-10
+amoxicillin 100 mg×2 on days 1-5
+clarithromycin 500 mg×2 on days 5-10
+tai-PPI (standard dose)×2 for all 10 days

### 6) L-cysteine/N-acetylcysteine

+bismuth subcitrate or subsalicylate 2×3-4
+tetracycline HCL 500 mg×4
+metronidazole or tinidazole 500 mg×2
+tai-PPI (standard dose)×2 for all 10 days
7) L-cysteine/N-acetylcysteine+chlarithromycin 500 mg×2+amoxicillin 1 g×2
8) Resistant strains:
a. L-cysteine/N-acetylcysteine+levofloxacin 500 mg+amoxicillin 1 g×2 for about 10 days.

Particularly, treatment is first attempted using the 1^{st} alternative, and the following one(s) are used if the previous one(s) has(have) not been successful.

Another alternative is to use a culture-guided antibiotic regimen.

According to a preferred alternative, the use of proton-pump inhibitors (PPIs) is avoided, as these decrease the acidity of the stomach, therefore potentially decreasing the natural capability of the acid and the pepsin in the stomach to react on the microbes or even directly on the biofilm. However, PPIs may improve the efficacy in some formulas by enhancing the availability of the active form of any of the antibiotics.

The present invention also concerns a treatment of patients suffering from biofilms formed by Helicobacter pylori or other microbes that are able to survive in the stomach, or several such microbes, including administering to said patients the above mentioned product comprising one or more compounds of the Formula I, containing one or more free sulphhydryl groups,

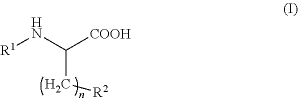

where
R¹ is hydrogen or an acetyl group,
R² is a sulphhydryl group or a C1-C5 straight chained or branched hydrocarbon chain, and
n is an integer of 1 to 3, preferably being 1, optionally containing one or more heteroatoms selected from O, N and S, further containing one or more free sulphhydryl groups, R² preferably being a sulphhydryl group, either as such or as combinations with antibiotics, whereby the compound(s) or optionally, when used, the antibiotic(s), are mixed with one or more pharmaceutically acceptable carriers providing sustained local release in the stomach.

The antibiotics are either mixed with the compound(s) of Formula I, or administered separately in a manner providing at least partial overlap of the period of action of the compound(s) of Formula I and the antibiotic(s) to obtain synergy.

The combination products and combination treatments of the present invention may also include administration of the compositions comprising one or more compounds of the Formula I and the antibiotics in connection with administering to the subject, or in connection with the subject consuming, further products having a suppressive effect on Helicobacter pylori, such as proton pump inhibitors, for example omeprazole, lansoprazole, pantoprazole or rabeprazole.

The invention and its advantages are further illustrated using the following example, which is not intended to limit the scope of the invention.

### EXAMPLES

### Example 1

### Eradication of Helicobacter pylori

In this open-label randomized trial, 50% of patients with at least four H. pylori eradication failures received before culture-guided antibiotic regimen 600 mg of N-acetylcysteine (NAC) once a day for seven days. In the NAC-pretreatment group H. pylori were successfully eradicated in 13/20 and in standard group only in 4/20 patients (p<01).

### Example 2

### Effect of L-Cysteine on Helicobacter pylori Biofilm Formation

In this example, H. pylori strains (NCTC 11637, National Culture Collection, London, UK, and ATCC 43504, American Type Culture Collection), grown in liquid culture without shaking, were examined to determine their ability to form biofilms in the presence and absence of L-cysteine.

The bacteria were screened for biofilm production using modified pellicle assay as previously described by Joshua et al. 2006 (14). The formation of pellicles at the liquid-gas interface of cultures grown without shaking was observed after 3-6 days.

All isolates were grown in 13-by 125-mm glass test tubes containing 6 ml of BHI broth supplemented with 2% (w/v) β-cyclodextrin (BCD; Sigma) in the presence and absence of L-cysteine. The bacterial concentration was adjusted equivalently to approximately 0.5 McFarland standard (˜10⁸ CFU/ml). The cultures were incubated under microaerobic conditions (N₂ 85%; O₂ 5%; CO₂ 10%) at 35° C. for 7 days without shaking. Development of pellicle and/or attached bacterial cells at interior surface of test tube at the air-liquid interface, representing as biofilm, was examined daily.

In the tests using L-cysteine and N-acetyl-cysteine, increasing amounts of L-cysteine or N-acetyl-cysteine were added in increasing concentrations to the culture medium.

Results (Inhibition of H. pylori Biofilm Formation by L-Cysteine and N-Acetyl-Cysteine):

10 mg/ml and 20 mg/ml of N-acetylcysteine and L-cysteine inhibited H. pylori biofilm formation completely (FIGS. 1 to 3), while 2 mg/ml of each substance inhibited biofilm formation partly.

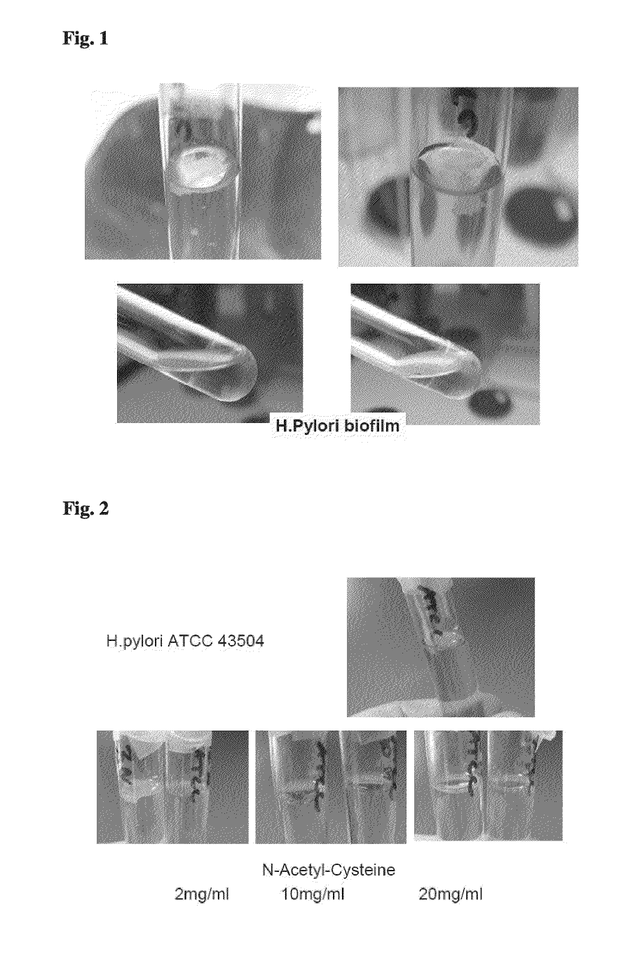

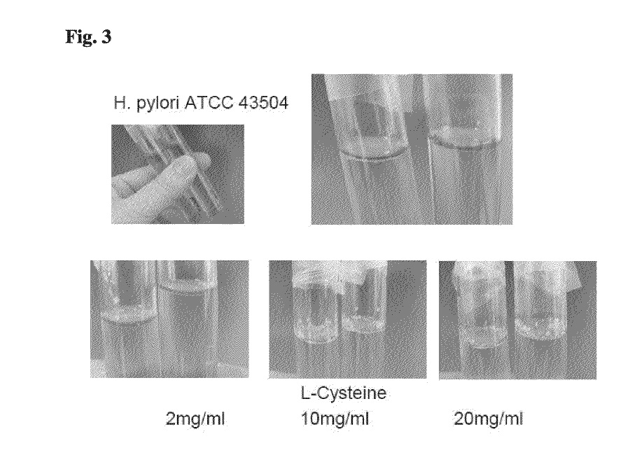

In FIG. 1, the pictures show a film adhered to the wall of the test tube, which film is the brown biofilm formed by H. pylori. This film is also visible in the upper picture of FIG. 2 and the upper right picture of FIG. 3, which illustrate the situation without the use of N-acetyl cysteine or L-cysteine.

Using L-cysteine (FIG. 3) or N-acetyl cysteine (FIG. 2), particularly when having added 10 mg/ml or 20 mg/ml of the active agent, no biofilm can be seen.

In studies with L-cysteine, a significant part of the L-cysteine was presumably oxidized to cystine and crystallized (FIG. 3). This suggests that smaller concentrations of L-cysteine are needed for the inhibition of H. pylori biofilm formation.

### REFERENCES

1. Malfertheiner P, Mégraud F, O'Morain C, Hungin A P, Jones R, Axon A, Graham D Y, Tytgat G. Current concepts in the management of Helicobacter pylori infection—the Maastricht 2-2000 Consensus Report. Aliment Pharmacol Ther 2002; 16: 167-180
2. Queiroz D M, Dani R, Silva L D, Santos A, Moreira L S, Rocha G A, Corrêa P R, Reis L F, Nogueira A M, Alvares Cabral M M, Esteves A M, Tanure J. Factors associated with treatment failure of Helicobacter pylori infection in a developing country. J Clin Gastroenterol 2002; 35: 315-320
3. Stark R M, Gerwig G J, Pitman R S et al. Biofilm formation by Helicobacter pylori. Lett Appl Microbiol 1999; 28:121-126.
4. Cole S P, Harwood L, Lee R et al. Characterization of monospecies biofilm formation by Helicobacter pylori. J Bacteriol 2004; 186:3124-3132.
5. Percival S L, Thomas J G. Transmission of Helicobacter pylori and the role of water in biofilms. J Water Health 2009; 7:469-477.
6. Canon M A, Tran V R, Sugawa C et al. Identification of Helicobacter pylori biofilms in human gastric mucosa. J Gastrointest Surg 2006; 10:712-717.
7. Coticchia J M, Sugawa C, Tran V R et al. Presence and density of Helicobacter pylori biofilms in human gastric mucosa in patients with peptic ulcer disease. J Gastrointest Surg 2006; 10:883-889.
8. Cammarota G, Branca G, Ardito F. et al. Biofilm demolition and antibiotic treatment to eradicate resistant Helicobacter pylori: A clinical trial. Clin Gastroenterol Hepatol 2010; 8:817-820.
9. Watanabe T, Ueno Y, Ogasawara A, Mikami T, Matsumoto T. Growth-inhibitory activity of Cladosporium cladosporioides by cysteine. Yakugaku Zasshi 2007; 127:1139-1144.
10. Salaspuro M P. Acetaldehyde, microbes and cancer of the digestive tract. Crit Rev Clin Lab Sci 2003; 40: 183-208.
11. Salaspuro V, Hietala J, Kaihovaara P, Pihlajarinne L, Marvola M, Salaspuro M.
Removal of acetaldehyde from saliva by a slow-release buccal tablet of L-cysteine. Int J Cancer. 2002 Jan. 20; 97(3):361-364.
12. Salaspuro V J, Hietala J M, Marvola M L, Salaspuro M P. Eliminating carcinogenic acetaldehyde by cysteine from saliva during smoking. Cancer Epidemiol Biomarkers Prev. 2006 January; 15(1):146-149.
13. Linderborg K, Marvola T, Marvola M, Salaspuro M, Färkkilä M, Väkeväinen S. Reducing carcinogenic acetaldehyde in the achlorhydric stomach with cysteine. Alcoholism Clin Exp Res, in press.
14. Joshua G W P, Guthrie-Irons C, Karlyshev A V, Wren B W. Biofilm formation in Campylobacter jejuni. Microbiology, 2006; 152: 387-396.

1-18. (canceled)

19. A composition comprising one or more compounds of the Formula I, containing one or more free sulphhydryl groups, 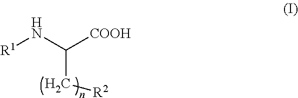
which are selected from cysteine or a cysteine derivative, and where
n is an integer of 1 to 3,
R¹ is hydrogen or an acetyl group, and
R² is a sulphhydryl group or a C1-C5 straight chained or branched hydrocarbon chain, optionally containing one or more heteroatoms selected from O, N and S, further containing one or more free sulphhydryl groups,
for use in partly or completely destroying biofilms formed by Helicobacter pylori or other microbes that are able to survive in the upper aerodigestive tract, or several such microbes, or for preventing such biofilm formation, or for eradicating the microbe(s), which compounds are mixed with one or more pharmaceutically acceptable carriers providing sustained local release in the stomach, the carrier being selected from the carriers that are capable of maintaining an effective concentration of the pharmaceutically active compound in the stomach for preventing the biofilm formation capacity of Helicobacter pylori that is able to survive in the upper aerodigestive tract, or several such microbes, during a period of 30 minutes, or destroying such a biofilm microbially formed on the surface of gastric mucosa.

20. The composition according to claim 19, characterized in that the compound of Formula I containing free sulphhydryl groups is L-cysteine or N-acetylcysteine.

21. The composition according to claim 19, characterized in that the carrier is selected from the carriers that are capable of providing an effective concentration of the pharmaceutically active compound in the stomach for eradicating Helicobacter pylori, or any other microbes that are able to survive in the upper aerodigestive tract, or several such microbes, when used as a mono therapy in patients infected with such microbes, or that are capable of causing an enhanced eradication rate of such microbes.

22. The composition according to claim 19, characterized in that it is for use in partly or completely destroying biofilms formed by Helicobacter pylori or any other microbes that are able to survive in the upper aerodigestive tract, or several such microbes, or for preventing such biofilm formation, or for eradicating the microbe(s), in patients carrying said microbe(s) that are resistant to one or more antibiotics that are used in the treatment of an infection by said microbe(s).

23. The composition according to claim 19, characterized in that the carrier is selected from carriers that are capable of controlling the releasing speed of the active compound(s) so that these compound(s) are released in the upper aerodigestive tract, particularly the stomach, during a period of 30-120 minutes, preferably 60-120 minutes.

24. The composition according to claim 19, characterized in that a single dose of the composition comprises 50-500 mg, preferably 50-300 mg, and most preferably 100-200 mg of the active compound(s) of Formula I, which dose can be provided using one or more formulations.

25. The composition according to claim 19, characterized in that the carrier comprises one or more substances, which are selected from the group of various chitosans, alginates, such as sodium alginate, aluminium hydroxide, sodium carboxymethyl cellulose, and sodium hydrogen carbonate, as well as enteric polymers, preferably at least one or more polymers from said group.

26. The composition according to claim 25, characterized in that it comprises 10-30%, preferably 20% sodium hydrogen carbonate of the weight of the polymers.

27. The composition according to claim 25, characterized in that the total amount of polymers in the composition is 10-50%, preferably 15-40%, and most preferably 20-30% of the weight.

28. The composition according to claim 19, characterized in that it comprises granules containing, as binders, enteric polymers, preferably methacrylate derivatives, the solution pH of which is 6-7 and the total amount of the weight of the preparation is 2-5%, preferably 3-4%.

29. The composition according to claim 19, characterized in that it is in the form of monolithic or multiparticular tablets or capsules, or granules as such, or it has the physical structure of a gel, preferably a tablet or a capsule comprising a mixture of powder or granules.

30. The composition according to claim 19, characterized in that it is in the form of capsules, which are intended to be administered to Helicobacter pylori infected patients daily for a period of from 3 days to 4 weeks, preferably for a period of 3 days to 3 weeks, more preferably 3 to 14 days.

31. The composition according to claim 30, characterized in that it is administered at 4 hours intervals 6 times a day, preferably to an empty stomach at least 1 hour, more preferably 2-5 hours after the previous meal.

32. Medical device for use in partly or completely destroying biofilms formed by Helicobacter pylori or other microbes that are able to survive in the stomach, or several such microbes, or for preventing such biofilm formation, or for eradicating the microbe(s), the device being in the form of monolithic or multiparticular tablets or capsules, or granules as such, or having the physical structure of a gel, characterized in the device being filled or mixed with a composition according to claim 20, optionally mixed with one or more further pharmaceutically acceptable carriers providing sustained local release in the stomach.

33. The medical device according to claim 32, characterized in that it is in the form of a tablet or a capsule.

34. The device according to claim 32, characterized in that the compound(s) of Formula I are used as a combination with one or more antibiotics selected from the group of amoxicillin, ampicillin, clarithromycin, metronidazole, tetracyclin and levofloxacin, either mixed with the compound(s) of Formula I, or administered separately in a manner providing at least partial overlap of the period of action of the compound(s) of Formula I and the antibiotic(s).

35. A combination comprising the composition according to claim 19 together with one or more antibiotics selected from the group of amoxicillin, ampicillin, clarithromycin, metronidazole, tetracyclin and levofloxacin, either mixed with the compound(s) of Formula I, or administered separately in a manner providing at least partial overlap of the period of action of the compound(s) of Formula I and the antibiotic(s), for partly or completely destroying biofilms formed by Helicobacter pylori or other microbes that are able to survive in the stomach or elsewhere in the upper aerodigestive tract, or several such microbes, or for preventing such biofilm formation, or for eradicating the microbe(s), which compound(s) and antibiotic(s) are mixed with one or more pharmaceutically acceptable carriers providing sustained local release in the stomach.

36. Treatment of patients suffering from biofilms formed by Helicobacter pylori or other microbes that are able to survive in the stomach or elsewhere in the upper aerodigestive tract, or several such microbes, including administering to said patients a composition according to claim 19.
